(19) United States
(12) Patent Application Publication
(10) Pub. No.: US 2015/0305989 A1
(43) Pub. Date: Oct. 29, 2015

(54) Methods and Products for Removing Sunscreen

(71) Applicant: Cristina Lupano, Santa Cruz, CA (US)

(72) Inventor: Cristina Lupano, Santa Cruz, CA (US)

(21) Appl. No.: 14/685,447

(22) Filed: Apr. 13, 2015

Related U.S. Application Data
(60) Provisional application No. 61/978,753, filed on Apr. 11, 2014.

(51) Int. Cl. A61K 8/02; A61K 8/37; A47K 7/02; A61K 8/89

(52) U.S. Cl. CPC A61K 8/0208; A61K 8/89; A61K 8/37; A47K 7/02

(57) ABSTRACT

The present invention provides for cloths comprising neoprene and methods of using such cloths to remove sunscreen. In particular the cloths of the present invention are suitable for use in a method for removing physical sunscreen from human skin. The cloth may have a nylon layer in addition to the neoprene layer and may be used in combination with a cleansing solution. Neoprene alternatives such as textiles that are comprised of at least some biorubber may be substituted for neoprene.

### FIELD OF THE INVENTION

The present invention relates to a cloth for removing sunscreen and methods of removing sunscreen. In particular, the present invention provides a textile substrate for removal of physical sunscreen.

### BACKGROUND OF THE INVENTION

The importance of wearing sunscreen has become increasingly apparent and public awareness about the characteristics and performance specifications of various forms of sunscreen has increased dramatically resulting in an increased appreciation and use of inorganic-based sunscreens, sometimes referred to as “physical” sunscreens. Inorganic-based sunscreens include ingredients capable of absorbing or scattering UV rays. Metal oxides having a high refractive index are frequently used. Numerous brands and forms of physical sunscreen are commercially available and are popular with people that are in the sun frequently and especially for those that participate in water sports such as surfing, sailing, swimming and the like. Physical sunscreens are often white, but can also come in a variety of colors, such as neon greens, pinks and such. ZINKA is a brand that is popular with surfers and comes in a variety of colors. Physical sunscreens may be sold in a variety of formulations and forms. Newer products including nanoparticles are available, but traditional physical sunscreens have been available for decades. U.S. Pat. No. 8,647,609 discloses a composite power form of inorganic-based sunscreens. US Patent Pub. No. 20080305133 discloses sunscreens comprising modified titanium dioxide particles.

Physical sunscreens that include compounds that deflect or block the sun's rays such as titanium dioxide, zinc oxide or other metal oxides tend to leave a white cast or tint to skin and do not remove easily, sometimes resulting in, for example, blocked pores, acne, discoloration of the skin and skin irritation.

Accordingly, there is a need in the art to develop improved methods and products for removal of skin products that include metal oxides or skin products that coat the surface of the skin and are relatively insoluble in water.

### SUMMARY OF THE INVENTION

The present invention provides cloths for removal of substances, such as physical sunscreens, that contain metal oxides. The cloth may have at least one layer of neoprene and optionally one or more layers of another fabric, for example, nylon or polyester.

In another aspect, methods for removing skin products that contain metal oxides are disclosed. Such methods may be wiping the cloth over the skin to remove the sunscreen or may optionally include the use of one or more cleansing solutions, gels or compounds.

In some aspects the cloth is provided in a form that provides for a pocket or strap for a human hand so that the cloth can be worn on a hand during use.

The cloth may also be modified with a grommet, hook, Velcro or other attachment to provide a means to attach the cloth to, for example, a wetsuit or to hang the cloth in a convenient location.

### BRIEF DESCRIPTION OF THE DRAWINGS

FIG. 1 shows an example of a cloth for removing sunscreen.

FIG. 2 shows an example of a cloth for removing sunscreen where the edges on 3 sides have been attached to form a pocket suitable for insertion of a hand so that the cloth may be worn like a mitt for sunscreen removal.

### DETAILED DESCRIPTION OF THE INVENTION

### Definitions and Nomenclature

It must be noted that, as used in this specification and the appended claims, the singular forms “a,” “an” and “the” include plural referents unless the context clearly dictates otherwise.

Unless defined otherwise, all technical and scientific terms used herein have the meaning commonly understood by one of ordinary skill in the art to which the invention pertains. Specific terminology of particular importance to the description of the present invention is defined below.

The term neoprene refers to a synthetic rubber produced by polymerization of chloroprene. Neoprene may also be referred to as polychloroprene. Neoprene fabrics have the characteristic that they shed water making them ideal for outdoor material. Neoprene is generally resistant to degradation from oil, solvents, sunlight, ozone, oxidation, rain, snow, sand and dust. The gas cells of neoprene make it an ideal insulation material frequently used for wetsuits and can holders, for example. Neoprene is preferably elastic and flexible, making it suitable for construction of form fitting products such as clothing and covers for objects of varying sizes and shapes, for example, camera equipment, electronics and sporting equipment. Neoprene can be constructed to have the appropriate thickness and density to absorb shock and thereby provide shock protection when used to construct covering for devices such as cameras, cell phones and as protection for the human body, for example, knee and elbow pads and braces for knees and elbows. Foamed neoprene containing gas cells is light weight, typically floats in water and can provide buoyancy to objects. Neoprene tolerates exposure to oils and various chemicals and maintains functionality over a wide temperature range. Neoprene is also resistant to abrasion. Neoprene can be produced in either a closed-cell form or an open-cell form. The closed-cell form is more waterproof, less compressible and generally more expensive than the open-cell form. The open-cell form is more breathable than the closed-cell form. Neoprene's burn point is around 260° C./500° F.

Wetsuits for outdoor sports such as surfing have historically been made from a neoprene material that can be laminated on one or two sides to fabric, such as polyester, spandex or nylon in a jersey knit. Material for the chamois may be purchased, for example, from Taiwanese neoprene manufacturers SHEICO, Inc. and NAM LIONG, Inc. which makes both petroleum and limestone based neoprene as well as biorubber based products, including guayule based products.

Neoprene fabric may be purchased commercially in a variety of forms and dimensions. Common thicknesses include 0.5 to 10 mm, with 1, 2, 3, 4, 5 and 6 mm being common. The product may have a fabric backing on one or both sides. When fabric is only included on a single side the non-fabric side may have a smooth or knobby (mesh) texture. The fabric may come in a variety of colors and prints

Neoprene is typically made by polymerizing chloroprene monomers into polymers. Fabrics that include at least a portion of an alternative for the rubber typically used to make neoprene may also be used in the cloth and methods of the present disclosure. The chloroprene monomers can be made using butadiene, derived from petroleum, or by dimerization of acetylene derived from limestone. Both processes have significant impacts on the environment either by requiring the use of a petroleum based product or the use of an energy intensive process to obtain the starting materials. There are alternative methods of making neoprene that require less fossil fuel, for example, neoprene can be made using biorubber. Examples of biorubber included rubbers that are made using the plant Guayule (Parthenium argentatum). Products that are made using Guayule have characteristics that provide advantages over other methods of making rubber. Methods for making synthetic rubber using Guayule are disclosed in U.S. Pat. No. 4,647,607. Guayule is a renewable, non-food crop that requires very little water, no pesticides and has a very clean manufacturing process compared to other processes for making traditional neoprene. YULEX Corporation makes a biorubber utilizing guayule and in 2012 released an alternative to the traditional neoprene wetsuit in partnership with PATAGONIA. YULEX's Closed Cell Foam (CCF) material is a guayule-based high performing replacement for polychloroprene, polyurethane, and ethylene vinyl acetate (EVA). The guayule-based CCF material can be produced in sheet fabric form with or without lamination. The rubber in the material contains about 60% guayule based rubber. A range of about 50 to 100% biorubber may be used. Additional advantages of the biorubber made using Guayule is that unlike traditional rubber it has a pleasant smell like eucalyptus or pine, instead of the petroleum smell often associated with neoprene. Other biorubbers may be made using the Russian dandelion (Taraxacum kok-saghyz).

Some people are allergic to neoprene or may develop dermatitis from residues left during production, for example, thiourea. A common accelerator used in the vulcanization of polychloroprene, ethylene thiourea (ETU) has been classified as a reprotoxic so in preferred embodiments the neoprene used in the present invention is not produced using ETU. Traditional rubbers used in neoprene can also contain proteins or other components that cause allergic reactions. Guayule does not produce these proteins and guayule based rubber contains no or a reduced amount of these proteins and would therefore be less likely to result in an allergic reaction for those individuals sensitive to neoprene made by other methods.

Other alternatives to traditional neoprene that may be used include ARIAPRENE™ a non-toxic synthetic rubber from Tiong Liong Corporation, Taiwan, and NEOGREEN® a formulation of neoprene that is free of phthalates, VOCs chlorine and metals that are found in traditional neoprene, made by GreenSmart, GrassValley, Calif. Additional fabrics that may be used in various embodiments include those described in U.S. Pat. No. 8,535,776.

The term cloth and wipe may be used interchangeably herein. Generally a cloth is used to refer to a piece of fabric of a given size and shape made out of materials suitable for the presently disclosed methods.

Cleaning cloths for solid surfaces have been disclosed, in for example, U.S. Pat. No. 5,591,507.

Compositions for removing eye make-up, including waterproof make-up are disclosed, for example, in U.S. Pat. Nos. 5,217,614 and 7,074,747, and may be used in combination with the cloth disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All patents and publications mentioned herein are incorporated herein by reference to disclose and describe the methods and/or materials in connection with which the publications are cited.

### Methods of the Invention

The present invention is directed generally to methods and products for removing products such as sunscreen from surfaces, particularly from skin. Physical sunscreens typically function by creating a physical barrier between the skin and potentially harmful UVA (320 to 400 nm) and UVB (290-320 nm) radiation or even against radiation of shorter (UV-C) or longer wavelength (IR). However, these sunscreens or components of these sunscreens can stick to the skin and are typically very difficult to remove effectively, causing acne by blocking skin pores and skin when the sunscreen is colored based. Most physical sunscreens are not easily removable. Rubbing the skin with soap does not effectively break down the mineral oil and beeswax used in many formulations but simply smears the compounds around without removing them substantially. Most of the mineral sunscreens are left on the skin even after a long warm shower. Physical sunscreens to which the methods are applicable may come in a variety of forms including, for example, paste, stick, lotion, cream and spray.

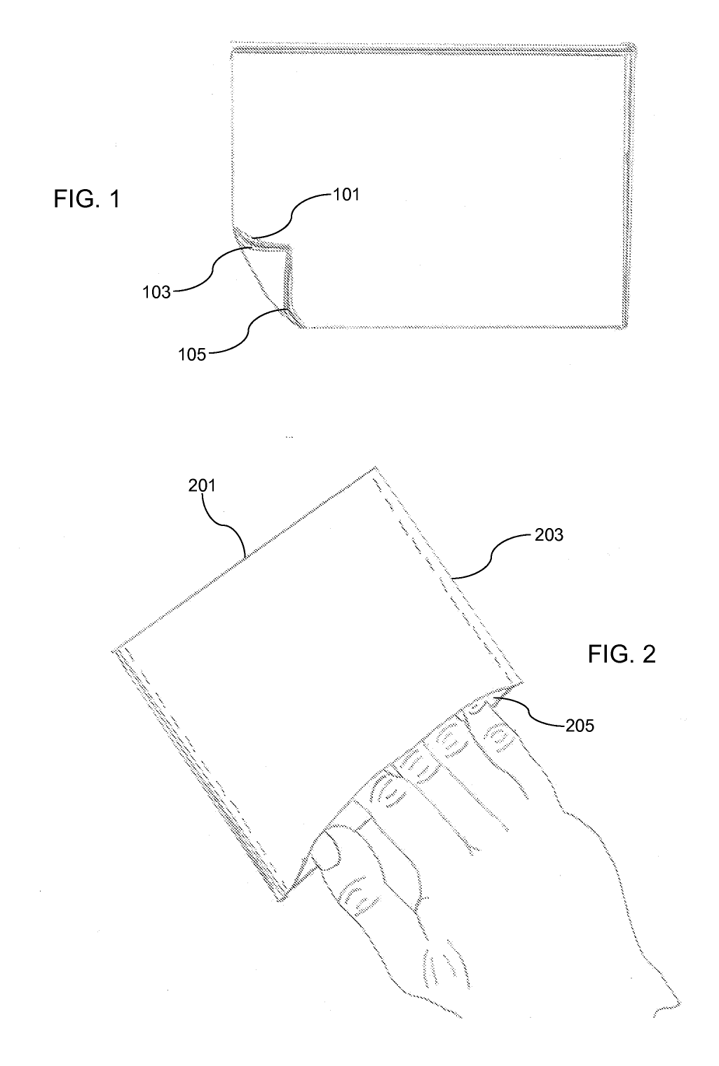

An example of a sunscreen removal cloth as disclosed herein is shown in FIG. 1. As shown the cloth has 3 layers, a first outer layer [101], a second outer layer [103] and an inner layer [105]. The inner layer [105] is preferably neoprene. The size of the cloth may vary but is generally larger than about 5 by 5 inches but smaller than about 18 by 18 inches. Preferred sizes may be, for example, 9 by 12 inches. The shape may be rectangular as shown, but it may also be provided in other shapes, for example, circular, oval, square, triangular or irregular in shape. The cloth may, for example, be cut in the shape of a surfboard or other shapes that evoke sport motifs. The outer layer(s) may be made of a fabric that has a printed design, for example, flowers, logos or surf or beach related imagery.

In another aspect the cloth may be folded substantially in half so form a pocket for a hand as shown in FIG. 2. The attachment of the edges [203] may be by sewing the edge on two sides as shown in the figure so that an opening [205] is left on one end and a folded edge [201] formed opposite the opening, but any method of attaching the edges may be used, for example gluing or a combination of available methods. FIG. 2 is not intended to be drawn to scale and the size of the hand relative to the dimensions of the cloth is not intended to be precise. For example, the cloth in preferred aspects would fit over the hand and cover the hand completely. In one aspect the cloth is folded substantially in half and is about the width and length of the hand so that it covers the hand up to about the wrist or beyond the wrist, but other sizes, shapes and folding patterns are also possible, for example the fold may be uneven so that less than half is folded over to form the upper portion of the pocket. In another aspect the cloth may be provided in the form of a mitten with a thumb region and a single region for the fingers of a hand.

In another embodiment a strap may be attached to one side of the cloth for ease of gripping. The hand may be placed between the strap and the cloth to attach the cloth to the hand. The strap may be of a stretchy or elasticized material so that it provides for snug fit on the hand.

In some embodiments the cloth can be used to remove diaper rash cream from skin. Many diaper rash creams contain metal oxides, for example, zinc oxide and like physical sunscreen may not remove easily with water or perspiration.

The cloth of the present invention is ideal for hand cleaning sunscreen from skin as it is less absorbent than materials such as cotton, but is pliable, smooth and stiff enough to provide substantial removal capability without being rough or abrasive to skin. The use of neoprene or biorubber or a combination thereof provides unexpected benefits over other fabrics, such as cotton or terry cloth. The neoprene and biorubber fabrics can also be washed in a standard washing machine with detergent and reused.

In some embodiments a cleaning agent may be applied to the surface of the cloth to assist in the removal of compounds from the skin. The cleansing agent is preferably one that imparts superior cleaning properties and is relatively non-irritating and thus suitable for use by people having sensitive skin and eyes. The cleansing agent may contain various combinations of the following: liquid silicone, one or more water dispersible components, an ester, water, a polymeric emulsifier and/or thickener, and a foaming surfactant. Such cleansing agents are disclosed, for example, in U.S. Pat. Nos. 7,074,747 and 8,263,059.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A cloth for removing sunscreen from the skin comprising a first layer comprising neoprene and a second layer comprising a woven fabric.

2. The cloth of claim 1, wherein the cloth is a fabric laminate of the first and second layers.

3. The cloth of claim 1, further comprising a third layer comprising a second woven fabric, wherein the first layer is between the second and third layers.

4. The cloth of claim 1, wherein the first layer comprises biorubber.

5. The cloth of claim 4, wherein the first layer is comprised of from about 40% to about 80% biorubber.

6. The cloth of claim 4, wherein the biorubber is produced from guayule.

7. The cloth of claim 5, wherein the biorubber is produced from guayule.

8. The cloth of claim 4, wherein the biorubber is produced from dandelion.

9. The cloth of claim 8 wherein the dandelion is taraxacum kok-saghyz.

10. The cloth of claim 8, wherein the first layer is comprised of from about 40% to about 80% biorubber.

11. The cloth of claim 1 wherein the cloth is folded substantially in half and sealed on two edges thereby forming a pocket.

12. The cloth of claim 1 wherein the woven fabric is nylon.

13. A method for removing sunscreen from the skin comprising wiping the skin with the cloth of claim 1.

14. The method of claim 13 further comprising using a cleansing composition in combination with the cloth.

15. The method of claim 14 wherein the cleansing composition comprises a liquid silicone, a water dispersible component and an ester.

16. A cloth for removing sunscreen from the skin comprising a first layer comprising biorubber and a second layer comprising a woven fabric.

17. The cloth of claim 16 wherein the first layer is comprised of 100% biorubber.

18. The cloth of claim 16 wherein the biorubber is made from guayule, dandelion or hevea or a combination thereof.

19. The cloth of claim 16 wherein the first layer comprises about 60% biorubber and about 40% neoprene.

20. The cloth of claim 19 wherein the biorubber is made from guayule.
